(19) United States
(12) Patent Application Publication
(10) Pub. No.: US 2021/0212564 A1
(43) Pub. Date: Jul. 15, 2021

(54) Head Harness & Wireless EEG Monitoring System

(71) Applicant: NEUROVIGIL, INC., Moffett Field, CA (US)

(72) Inventors: Philip Low, La Jolla, CA (US); Yu M. Chi, La Jolla, CA (US); Siddharth Joshi, La Jolla, CA (US); Christopher Uebelher, San Diego, CA (US); Yvon A. Dubois, Escondido, CA (US); Kevin Liu, Walnut, CA (US)

(21) Appl. No.: 17/214,574

(22) Filed: Mar. 26, 2021

Related U.S. Application Data
(63) Continuation of application No. 12/913,686, filed on Oct. 27, 2010.
(60) Provisional application No. 61/255,343, filed on Oct. 27, 2009.

(51) Int. Cl. A61B 5/00; A61B 5/291; A61B 5/369

(52) U.S. Cl. CPC A61B 5/0006; A61B 5/6803; A61B 5/274; A61B 5/291; A61B 5/369; A61B 5/6831

(57) ABSTRACT

An assembly and system for collection and assessment of physiological data is provided. The assembly includes a physiological data acquisition module that may be used in combination with a head harness for the collection, recordation, storage and transmission of quality physiological data. The assembly integrates easy to use, self-applied electrodes in a user-friendly system resulting in less data artifacts than commonly seen in conventional methods and techniques for collecting physiological data. The assembly and system captures high-quality physiological data for display, storage, processing and analysis.

### CROSS-REFERENCE TO RELATED APPLICATIONS

This application is a continuation of U.S. patent application Ser. No. 12/913,686 filed Oct. 27, 2010, now pending; which claims the benefit under 35 USC § 119(e) to U.S. Provisional Patent Application Ser. No. 61/255,343 filed Oct. 27, 2009, now expired. The disclosure of each of the prior applications is considered part of and is incorporated by reference in the disclosure of this application.

### BACKGROUND OF THE INVENTION

### Field of the Invention

The present invention relates generally to devices for the acquisition of physiological data. More particularly the present invention is directed to a physiological data acquisition assembly that may be used in combination with a head harness, applied by a user without assistance and that acquires quality physiological data including but not limited to EEG, EKG, EMG, and EOG signals.

### Background Information

An electroencephalogram (EEG) is the graphic record of brain action potentials. The EEG supplies important information about the brain function of a patient. Conventional monitoring and diagnostic equipment is structured in such a way that several electrodes are mounted on the subject, which tap the brain signals and transmit the signals via cables to amplifier units. Normally, separate electrodes are used for each measurement parameter and usually require that numerous electrodes be placed on a patient's head. Proper placement of the electrodes is important and usually follows the International 10/20 System, which is the widely accepted method describing the placement of the electrodes on the patient's head. As a result, EEG data is often gathered in a clinical setting where the electrodes can be properly placed with technical assistance.

Due to the nature of the conventional methods applied in EEG examinations, many cables are suspended on the patient. The cables are troublesome in that they constrict the patient and greatly limit the freedom of movement. In addition, the process of properly placing numerous electrodes is time consuming and often, due to the number of wires and electrodes, will result in ineffective adhesion and loss of contact with the scalp of the patient.

These conventional methods are often a problem in monitoring patients in sleep centers. The complications associated with the cumbersome nature of the conventional detection and recording devices coupled with the subject being in a foreign environment greatly hinders monitoring a patient's sleep stage. The combination of the cables and sleep center environment will have a bearing on the patient's ability to sleep and subsequent results of the exam. If a subject has the ability to be monitored comfortably from home without restricting their freedom of movement, anxiety is reduced therefore producing more accurate results.

There is therefore a need for a new and improved physiological data acquisition system and apparatus that facilitates application of electrodes by the patient without technical assistance and that collects quality physiological data, including but not limited to EEG, EKG, EMG, and EOG signals from a single active electrode.

### SUMMARY OF THE INVENTION

The present invention provides for a system and assembly for acquiring physiological data that can be applied by a user without technical assistance and acquire quality physiological data including but not limited to EEG, EKG, EMG, and EOG signals. The present invention is primarily used in conjunction with sleep centers for the examination, diagnoses and treatment of sleep disorders.

In a preferred embodiment of the present invention, a physiological data acquisition assembly for use in combination with a head harness is provided. The physiological data acquisition assembly comprises a physiological data acquisition module adapted to be removably housed by the head harness, wherein the head harness includes a base strap adapted to be adjustably secured around the circumference of a user's head, an upper portion housing the physiological data acquisition module and a plurality of longitudinally extending straps for detachably securing the housing for the physiological data acquisition module to the base strap. The physiological data acquisition assembly also comprises at least one electrode snap connector assembly for use on an active electrode and at least one electrode snap connector assembly for use on a biased ground electrode.

According to another embodiment of the present invention, a physiological data acquisition assembly is provided comprising, at lease a single channel of physiological data wherein at least one active electrode and at least one reference electrode are in close proximity. The physiological data acquisition assembly also comprises a singular sensor patch wherein the at least one active electrode and at least one reference electrode are located but not electrically coupled.

According to yet another embodiment of the present invention, a system for acquiring physiological data is provided comprising a physiological data acquisition module coupled to at least one electrode snap connector assembly for use on an active electrode and at least one electrode snap connector assembly for use on a reference electrode. The system also comprises a head harness for housing the physiological data acquisition module, wherein the head harness includes a base strap adapted to be adjustably secured around the circumference of a user's head, an upper portion housing the physiological data acquisition module and a plurality of longitudinally extending straps for detachably securing the housing for the physiological data acquisition module to the base strap. The system also comprises a means for displaying, storing, processing and analyzing data transmitted by the physiological data acquisition module.

According to yet another embodiment of the present invention, a system for acquiring physiological data is provided comprising at least a single channel of physiological data wherein at least one active electrode and at least one reference electrode are in close proximity. The system also comprises a singular sensor patch wherein the at least one active electrode and the at least one reference electrode are located but not electrically coupled. The system also comprises a means for displaying, storing, processing and analyzing encrypted data transmitted by the physiological data acquisition module.

It is contemplated that any method, system or information described herein can be implemented with respect to any other method, system or information described herein.

Unless otherwise defined, all terms used herein have the same meaning as commonly understood by one of ordinary skill in the art to which this invention belongs. Methods and materials are described herein for use of the present invention; other suitable methods and materials known in the art can also be used. The materials and methods, and examples are illustrative only and not intended to be limiting. All publications, patent applications, patents and other references mentioned herein, are incorporated by reference in their entirety. In case of conflict, the present specification, including definitions will control.

These and other embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE FIGURES

The present invention, both as to its organization and manner of operation, together with further objects and advantages, thereof, may best be understood with reference to the following description, taken in connection with the accompanying drawings in which:

FIG. 1 shows a drawing of an exploded perspective view of the physiological data acquisition assembly for use in combination with a head harness.

FIG. 2 shows a drawing of a front perspective view of the physiological data acquisition assembly for use in combination with a head harness as applied to a patient.

FIG. 3 shows a flowchart of the system according to the present invention. The device contains the components below without limiting the general idea of the invention.

### DETAILED DESCRIPTION OF THE INVENTION

The present invention is based on the ability to monitor physiological data for the study of brain performance during the normal course of daily activities including but not limited to sleep, in the past, has required cumbersome detection and analysis equipment and in many instances has required the need for technical assistance. The present invention disclosed herein provides a benefit over existing conventional methods by allowing the collection of data related to a physiological event from the comfort of the patient's home. The present invention employs a novel physiological data acquisition assembly and easy to follow system that is user friendly and overcomes the disadvantages of the conventional monitoring methods.

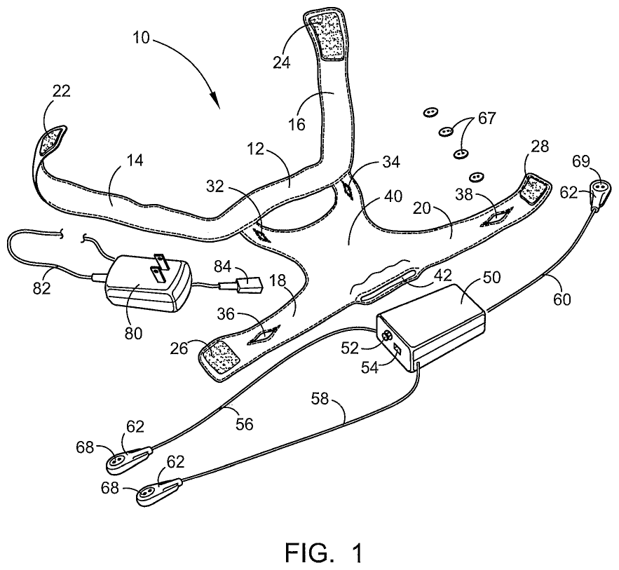

Referring to FIG. 1, an exploded perspective view of the physiological data acquisition assembly for use in combination with a head harness 10 is depicted. The head harness 10 may house one or more devices or wires. The head harness is worn over the head and hair of a patient. The head harness includes a front pad 12 having a first end 14 and second end 16. The front pad 12 is adapted to extend across the patient's forehead. The first end 14 and second end 16 are adapted to be adjustably secured around the circumference of a user's head using a fastener preferably a hook 22 and loop 24. In another embodiment, the fastener may be a hook and loop, Velcro, snap, button, buckle or any other fastening device that allows for custom fit, adjustment and comfort.

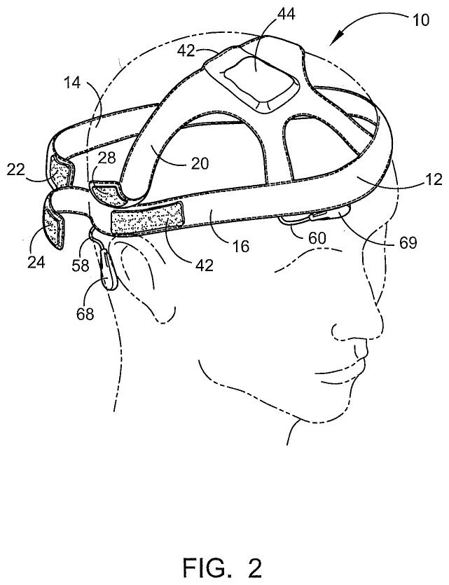

The head harness also includes an upper portion 40 including longitudinally extending straps 18 and 20 for detachably securing the upper portion 40 to the base strap 12, 14, & 16. The longitudinally extending straps 18 and 20 are detachably secured to the base strap at 14 & 16 using a fastener, preferably a hook 26 & 28 and loop 42, FIG. 2. In another embodiment, the fastener may be a hook and loop, Velcro, snap, button, buckle or any other fastening device that allows for custom fit, adjustment and comfort.

In a preferred embodiment, the head harness may be made of one or more layers of material to create a hollow core through all parts of the harness. The physiological data acquisition module 50 is removably received in the hollow core 42 & 44, FIG. 2 of the upper portion 40. In this preferred embodiment, the upper portion 40 and longitudinally extending straps 18 & 20 include a plurality of slots 32, 34, 36 & 38 for receiving the electrode snap connector assemblies 68 & 69 and associated lead wires 56, 58 & 60 coupled to the physiological data acquisition module 50.

The electrode snap connector assemblies 68 represent a combination of a biased ground electrode and a reference electrode snap connectors and in the preferred embodiment the electrodes 67 are placed behind the left and right ear of the patient, FIG. 2. The biased ground electrode snap connector assembly and the reference electrode snap connector assembly 68 may be color coded to distinguish the two. In another embodiment of the present invention, only a reference electrode snap connector assembly is required for the acquisition of electrical physiological data. The electrode snap connector assembly 69 represents the active electrode snap connector and in the preferred embodiment the electrode 67 is placed on the forehead of the patient. The active electrode and reference/biased ground electrodes 67 may be a self-adhesive conductive electrode, a wet, dry, contact, non-contact or EKG electrode. The electrode snap connector assemblies also include a noise reducing or cancelling amplifier 62 at the electrode connection level to reduce any electrical noise that may be picked up by the lead wires 56, 58 & 60. To further improve the performance of the physiological data acquisition module 50, the module or the electrode snap connector assemblies 68 & 69 are configured to continuously monitor electrode impedance and may include lights indicative of the current status of the integrity of the electrode contacts.

In another embodiment of the present invention, both active and reference electrodes 67 are placed in close proximity with respect to each other but are not electrically connected. In this embodiment, the active and reference electrodes are located on a singular sensor patch.

In a preferred embodiment, the physiological data acquisition module 50 includes a battery power component that includes a rechargeable small form factor, high capacity battery. The physiological data acquisition module 50 includes a power supply and recharging circuitry for receiving power through an electrical power cord 82 and AC unit 80. The electrical power cord 82 is coupled to the physiological data acquisition module for recharging the small form factor, high capacity battery through a port 54, which may be but is not limited to USB, DB-25 or the like. The physiological data acquisition module 50 includes a power on and off function 52 for preserving the power supply of the small form factor, high capacity battery when not in use. The physiological data acquisition module 50 may also include power on and off indicator lights indicative of the current status of the physiological data acquisition module 50. In another embodiment, the physiological data acquisition module rechargeable small form factor, high capacity battery may be recharged through a USB connection to a computer.

The physiological data acquisition module 50 is configured to record, transmit and store encrypted data collected from the electrode snap connector assembly 69 for use on an active electrode 67 applied to the forehead region of a patient. The electrode snap connector assemblies 68 for use on a reference and biased ground electrode 67 are placed behind the ears of the patient. The biased ground electrode 67 functions to stabilize the baseline and improve immunity from external interferences.

In a preferred embodiment, the physiological data acquisition module 50 is configured to include a wireless transmitter/receiver for transmitting wirelessly the recorded and stored encrypted data to a remote center or computer for further display, storing, processing and analysis or for transmitting wirelessly in real time the encrypted data to a remote center or computer for further display, storing, processing and analysis. In another aspect of the present invention, the recorded and stored data may be transmitted wirelessly to a device including but not limited to a cellular telephone, smart-phone, iPad® and/or computer. The wireless transmitter/receiver may also be included on the singular sensor patch to transmit wirelessly to a device including but not limited to a cellular telephone, smart-phone, iPad® and/or computer. The recorded and stored data may also be transmitted directly to a computer, cellular telephone, smart-phone and/or iPad® via USB transfer capabilities incorporated at port 54 of the physiological data acquisition module 50.

Referring now to FIG. 2, a front perspective view of the physiological data acquisition assembly for use in combination with a head harness as applied to a patient is depicted. In a preferred embodiment, the physiological data acquisition module 50 is housed in a hollow cavity 44 of the upper portion 40 of the head harness. In another embodiment, the physiological data acquisition module 50 is removably affixed to the head harness by a fastener that may be a hook and loop, Velcro, snap, button, buckle or any other fastening device that allows for custom fit, adjustment and comfort.

In yet another embodiment of the head harness there may be openings that allow access to the interior of the harness as well as allow for connections to be made from the interior of the harness and exterior components. In another embodiment the design may be independent of any specific device or wire purpose other than those listed here. The head harness allows any devices or wires or electronic components to be removed for service, replacement or safety. The head harness may be washable, cleaned or sterilized. The head harness may be disposable, independent of the devices or wires housed.

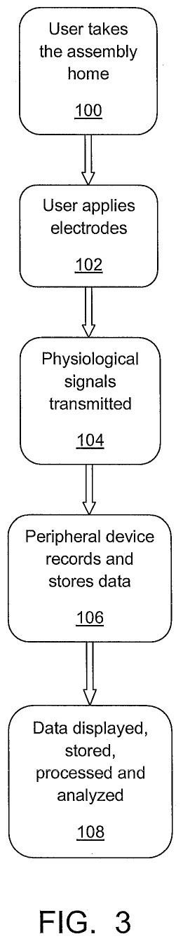

Referring now to FIG. 3, a flowchart of the system according to the present invention is depicted. In the preferred embodiment, a user is supplied with the invention and allowed to use its application in the home 100. It is to be understood that the nature of the present invention allows the user to apply the device in any setting and is not limited to home or clinical use. At 102, due to the ease of application, the user applies at least one electrode designated the active electrode and at least one electrode designated the reference electrode. In one embodiment, the active and reference electrode may be applied to the forehead and behind the ear respectively. In another embodiment, the active electrode is applied to the forehead while a reference electrode and a biased ground electrode are applied behind the ears of the user. In yet another embodiment, the active electrode and reference electrode are contained in close proximity on a singular sensor patch and applied to the head of the user. It is to be understood that the application of the electrodes may or may not be used in combination with a head harness.

Once the electrodes have been placed by the user, physiological electrical data is collected. At 104, the physiological data is transmitted either wirelessly by a physiological data acquisition module 50 or is transmitted wirelessly directly from the singular sensor patch to a peripheral device that may be but is not limited to a computer, cellular telephone, smart-phone and/or iPad®. The peripheral device is configured to record and store the data 106. Further, the peripheral device is configured to display, store, process and analyze 108 the transmitted encrypted data. The means for displaying, storing, processing and analyzing may be but is not limited to a computer, cellular telephone, smart-phone and/or iPad® or any other remote display device.

In certain alternative embodiments of the present invention, the assembly and system are configured to accommodate more than one channel of physiological data. For example and not by way of limitation, the assembly and system incorporate sensors, i.e., head position sensor, airflow sensor using acoustics, nasal pneumotachometer, body temperature sensor and oximeter, alone or in various combinations for collecting data.

The assembly and system may also be in communication with a remote control device. The remote control device may function as a gateway device to other peripheral devices. In this capacity, the remote control device is configured to record and store encrypted data transmitted by the assembly and system, monitor the small form factor, high capacity battery life and recorded and stored data levels maintained by the physiological data acquisition module. Further, the remote control device in its capacity as a gateway device may transmit and receive recorded and stored encrypted data either through a wired or wireless connection with a peripheral device for display, storage, processing and analysis.

Systems and materials are described herein. However, systems and materials similar or equivalent to those described herein can be also used to obtain variations of the present invention. The materials, systems, and examples are illustrative only and not intended to be limiting.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The previous description of some aspects is provided to enable any person skilled in the art to make or use the present invention. Various modifications to these aspects will be readily apparent to those skilled in the art, and generic principles defined herein may be applied to other aspects without departing from the spirit or scope of the invention. For, example one or more elements can be rearranged and/or combined, or additional elements may be added. Thus, the present invention is not intended to be limited to the aspects shown herein but is to be accorded the widest scope consistent with the principles and novel features disclosed herein.

Although the invention has been described with reference to the above example, it will be understood that modifications and variations are encompassed within the spirit and scope of the invention. Accordingly, the invention is limited only by the following claims.

What is claimed is:

1. A physiological data acquisition assembly for use in combination with a head harness, the physiological data acquisition assembly comprising:
a physiological data acquisition module adapted to be removably housed by the head harness, wherein the head harness includes a base strap adapted to be adjustably secured around the circumference of a user's head, an upper portion housing the physiological data acquisition module and a plurality of longitudinally extending straps for detachably securing the housing for the physiological data acquisition module to the base strap;
at least one electrode snap connector assembly for use on an active electrode; and
at least one electrode snap connector assembly for use on a reference electrode.

2. The physiological data acquisition assembly of claim 1, wherein the physiological data acquisition module further comprises at least one electrode snap connector assembly for use on a biased ground electrode.

3. The physiological data acquisition assembly of claim 2, wherein the electrode snap connector assemblies include a noise reducing or cancelling amplifier.

4. The physiological data acquisition assembly of claim 2, wherein the active electrode, reference electrode and biased ground electrode is a self-adhesive conductive wet, dry, contact or non-contact electrode.

5. The physiological data acquisition assembly of claim 1, wherein the physiological data acquisition module is configured to record, transmit and store encrypted data collected from the active electrode.

6. The physiological data acquisition assembly of claim 5, wherein the physiological data acquisition module includes at least one port to charge the module, transmit and receive data.

7. The physiological data acquisition assembly of claim 6, wherein the physiological data acquisition module further comprises a RF transmitter-receiver for transmitting encrypted data to a remote center or computer for further display, storing, processing and analysis.

8. The physiological data acquisition assembly of claim 7, wherein the recorded and stored encrypted data is transmitted wirelessly to a device selected from at least one member of the group consisting of a cellular telephone, smart-phone and computer.

9. The physiological data acquisition assembly of claim 8, wherein the RF transmitter-receiver is in wireless communication with a remote control.

10. The physiological data acquisition assembly of claim 9, wherein the remote control acts as a power source or docking station.

11. The physiological data acquisition assembly of claim 1, wherein the physiological data acquisition module includes additional components selected from at least one member of the group consisting of a head positioning sensor, nasal pneumotachometer, body temperature sensor and oximeter.

12. A physiological data acquisition assembly, the physiological data acquisition assembly comprising:
at least a single channel of physiological data wherein at least one active electrode and
at least one reference electrode are in close proximity; and
a singular sensor patch wherein the at least one active electrode and at least one reference electrode are located but not electrically coupled.

13. The physiological data acquisition assembly of claim 12, wherein the singular sensor patch includes a RF transmitter-receiver for transmitting encrypted data to a remote center or computer for further display, storing, processing and analysis.

14. The physiological data acquisition assembly of claim 13, wherein the wirelessly transmitted encrypted data is to a device selected from at least one member of the group consisting of a cellular telephone, smart-phone and computer.

15. A system for acquiring physiological data, the system comprising:
a physiological data acquisition module coupled to at least one electrode snap connector assembly for use on an active electrode and at least one electrode snap connector assembly for use on a reference electrode;
a head harness for housing the physiological data acquisition module, wherein the head harness includes a base strap adapted to be adjustably secured around the circumference of a user's head, an upper portion housing the physiological data acquisition module and a plurality of longitudinally extending straps for detachably securing the housing for the physiological data acquisition module to the base strap; and
a means for displaying, storing, processing and analyzing encrypted data transmitted by the physiological data acquisition module.

16. The system of claim 15, wherein the encrypted data transmitted by the physiological data acquisition module is transmitted by a RF transmitter-receiver to the means for displaying, storing, processing and analyzing.

17. A system for acquiring physiological data, the system comprising:
a single channel of physiological data wherein at least one active electrode and at least one reference electrode are in close proximity;
a singular sensor patch wherein the at least one active electrode and at least one reference electrode are located but not electrically coupled; and
a means for displaying, storing, processing and analyzing encrypted data transmitted by the physiological data acquisition module.

18. The system of claim 17, wherein the singular sensor patch includes a RF transmitter-receiver for transmitting encrypted data to the means for displaying, storing, processing and analyzing.
